(19) United States
(12) Patent Application Publication
(10) Pub. No.: US 2025/0295858 A1
(43) Pub. Date: Sep. 25, 2025

(54) Universal Plunger Rod and Method for Connecting and Disconnecting the Plunger Rod and a Plunger Stopper

(71) Applicant: Becton Dickinson France, Le Pont-de-Claix (FR)

(72) Inventors: Nastasja Grillet, Cusy (FR); Julien Gagliano, Meylan (FR); Anne Milleret, Pontcharra (FR)

(21) Appl. No.: 18/997,151

(22) PCT Filed: Jul. 18, 2023
(86) PCT No.: PCT/EP2023/069894

§ 371 Date: Jan. 20, 2025

(30) Foreign Application Priority Data
Jul. 21, 2022 (EP) 22306088.0

(51) Int. Cl. A61M 5/315

(52) U.S. Cl. CPC A61M 5/31515

(57) ABSTRACT

A plunger rod to be connected to a plunger stopper, including a rod, a sleeve having a movable part capable of moving from a first radial position to a second radial position under the effect of a centrifugal force applied thereon. Said rod being capable of sliding in translation within said sleeve from a first axial position to a second axial position under the effect of a distal pressure exerted on its proximal end. Releasable locking means configured for temporary locking said rod in its second axial position. The invention also relates to a method for connecting/disconnecting the plunger rod and a plunger stopper.

### CROSS-REFERENCE TO RELATED APPLICATIONS

This application is the United States national phase of International Patent Application No. PCT/EP2023/069894, filed Jul. 18, 2023, and claims priority to European Patent Application No. 22306088.0, filed Jul. 21, 2022, the disclosures of which are hereby incorporated by reference in their entireties.

### BACKGROUND OF THE INVENTION

### Field of the Invention

The present invention relates to a plunger rod intended to be connected to a plunger stopper in view of expelling or reconstituting a product, such as drug, present within a container of an injection device.

In this application, the distal end of a component or of a device means the end furthest away from the hand of the user and the proximal end means the end closest to the hand of the user, when the component or device is in the use position. Similarly, in this application, the terms “in the distal direction” and “distally” mean in the direction of the injection, and the terms “in the proximal direction” and “proximally” mean in the direction opposite to the direction of injection.

Injection devices, such as syringes, are well known. Many different types of injection devices have been designed for administering medicines. Injection devices usually comprise a container intended to receive the medicines or product to be injected and a plunger rod intended to move a plunger stopper within the container. The plunger stopper may be moved in the distal direction to expel a product at the time of injection. Alternatively, the plunger stopper may be moved in the proximal direction to withdraw a product for example from a separate vial and transfer it within the container of the injection device. Empty and pre-filled disposable injection devices exist on the market, and are convenient, safe and efficient and may be used directly in emergency cases.

Prefilled injections devices are filled with a medicine, such as a pharmaceutical drug, packaged for use, and then typically stored at a doctor's office, hospital, etc. until they are needed for use. In order to ensure that the product present within the container will not leak outwards during the storage, the container is closed at its distal end by a rubber cap, and at its proximal end by a plunger stopper.

In some cases, a plunger rod is connected to the plunger stopper of the prefilled injection device. Nevertheless, in such a condition, the prefilled injection device will take up a significant amount of storage space. Required storage space is an important feature for prefilled injection devices. It may be especially important when the medicine contained in these devices must be stored and transported at low temperatures.

In hospitals and pharmacies, storage space for medicines is limited. It is thus desirable that the prefilled injection devices occupy as less volume as possible during transportation and/or storage.

In this view, prefilled injection devices are more and more provided without any plunger rod, the proximal end of the injection device being sealed by the plunger stopper on its own. In such cases, separate plunger rods are provided, and the plunger stopper is usually provided with connecting means intended to cooperate with complementary connecting means located on the plunger rod, in order to connect the plunger stopper and the plunger rod together when time has come to proceed with the injection. In a conventional manner, these connecting means are cooperating threads or hooks: the proximal end of the plunger stopper is provided with a cavity having an inner thread and the distal end of the plunger rod is provided with an outer thread capable of cooperating with the inner thread of the cavity of the plunger stopper in order to screw the plunger rod into the plunger stopper.

### Description of Related Art

Anyway, with the development of medicines and of injection device manufacturers all over the world, the designs and dimensions of the inner threads of the plunger stoppers as well as the material forming the plunger stopper may vary and it may happen that some plunger rods are not compatible with the plunger stoppers sealing the proximal ends of prefilled injection devices. The incompatibility between the plunger rod and the plunger stopper may cause bending and/or breakage of the plunger rod during the screwing step of the plunger rod into the plunger stopper and/or may impact container closure integrity and the injection of the product cannot take place in a safe manner. Also, for two not compatible plunger stopper and plunger rods, withdrawal may be impossible and reconstitution cannot be executed properly.

There is therefore a need for a plunger rod that would be compatible with any plunger stopper, in such a way that the user would feel confident that the connection between the plunger rod and the plunger stopper is effective and secured, whatever the specific feature, the design and/or the dimensions of the connecting means, such as the inner thread or hook space, of the cavity of the plunger stopper, and whatever the material the plunger stopper may be made of.

Moreover, plunger rods are usually made of plastic. Plunger rods are generally single used and discarded with the prefilled injection device once the injection is completed. Plastic plunger rods contribute to the global plastic waste generated by the sector of medical devices.

There is therefore the further need for a plunger rod that, not only would be capable of realizing a safe connection with any plunger stopper of an injection device whatever the design and/or material of such a plunger stopper, but that in addition would be removable from said plunger stopper and would be reusable with the plunger stopper of another prefilled injection device, as many times as possible.

### SUMMARY OF THE INVENTION

A first aspect of the invention is a plunger rod intended to be connected to a proximal cavity of a plunger stopper of an injection device, said plunger rod comprising:
A rod having a proximal end and a distal end,
A sleeve configured to receive at least the distal end of the rod, a distal portion of said sleeve being provided with at least one movable part capable of moving from a first radial position to a second radial position under the effect of a force applied on said movable part,
Said rod being capable of sliding in translation within said sleeve from a first axial position, in which the distal end of said rod is not in contact with said movable part, said movable part is in its first radial position and the distal portion of said sleeve may freely enter in, and exit from, said proximal cavity of said plunger stopper, to a second axial position, in which the distal end of said rod has applied a force on said movable part to move said part in its second radial position, and said movable part is capable of harpooning an inner wall of said proximal cavity of said plunger stopper to connect said plunger rod to said plunger stopper, said rod being configured to translate from its first axial position to its second axial position under the effect of a distal pressure exerted on its proximal end,
Releasable locking means configured for temporary locking said rod in its second axial position.

In embodiments, the plunger rod may further comprise biasing means configured for automatically moving said rod from its second axial position to its first axial position when said releasable locking means are released.

The plunger rod of the invention may be connected to the proximal cavity of a plunger stopper whatever the design of the inner wall of said cavity. In particular, the inner wall of the proximal cavity may be provided with a thread of any dimensions or design, the movable part of the plunger rod of the invention will harpoon the inner wall of the cavity when moving from its first radial position to its second radial position, and will therefore attach the plunger rod to the plunger stopper by means of interference fit or by hooking, and not by means of screwing. The plunger rod of the invention is therefore not screwed into the plunger stopper, but it is fixed into the plunger stopper by means of the movable part harpooning the inner wall of the cavity. Indeed, plunger stoppers are usually made of elastomeric material, such as rubber, and the movable part can easily harpoon a wall made of such a material.

The plunger rod of the invention therefore allows simplifying the connecting step between the plunger rod and the plunger stopper. In particular, thanks to the plunger rod of the invention, the end-user no longer needs to screw the plunger rod into the plunger stopper. The risk that the plunger rod bend and/or break during the connecting step with the plunger stopper is in consequence significantly reduced. The plunger rod of the invention further allows removing one processing step in the case where the plunger rod is assembled with high speed machine at customer site.

In addition, thanks to the plunger rod of the invention, it is possible to use a single plunger rod for injection devices having plunger stoppers made of different materials and/or provided with connecting means, such as inner threads, having different designs. The plunger rod of the invention therefore helps streamlining the packaging, transportation and storage process of prefilled injection devices, as well as the use of such prefilled injection devices by the end-user within healthcare premises such as hospitals.

In addition, the plunger rod of the invention may easily be disconnected from a plunger stopper to which it has been attached as described above. Thanks to the biasing means of the plunger rod of the invention, the movable part may be easily detached from the inner wall of the cavity in which it is fixed. The movable part is allowed to move back to its first radial position, where the rod may be easily removed from the proximal cavity of the plunger stopper. Therefore, the end-user does not need to perform an unscrewing step in order to disconnect the plunger rod from the plunger stopper. Thanks to the plunger rod of the invention, the disconnection step is rendered very simple.

The plunger rod of the invention therefore further helps reducing the global plastic waste generated by the medical sector and is therefore environment friendly. Indeed, thanks to the plunger rod of the invention, it is possible to reuse the same plunger rod for a plurality of prefilled injection devices, thereby saving a significant amount of plunger rods and avoiding unnecessary plastic waste.

In embodiments, the proximal end of said rod is being provided with a button cap, and a proximal portion of the sleeve is being provided with a radial flange, and wherein said biasing means comprise a coil spring abutting on both a proximal surface of said radial flange and on a distal surface of said cap, said coil spring being capable of transitioning from a rest position where said rod is in its first axial position, to a stressed state where said rod is in its second axial position. Such embodiments allow performing the connection of the plunger rod to the plunger stopper in a simple manner, as the end user simply has to push distally on the cap of the plunger rod to realize the connection.

In embodiments, said rod being configured to rotate with respect to said sleeve, said releasable locking means comprise at least one hook extending distally from said cap and at least one partial circumferential groove located on said radial flange, said hook and said partial circumferential groove being configured to:
engage one another to lock said rod in its second axial position when one exerts a distal pressure on said cap so as to put said coil spring in its stressed state, and further rotates said rod with respect to said sleeve in a first direction, and
disengage from one another to free said rod from its second axial position and move it automatically to its first axial position, when one rotates said rod with respect to said sleeve contrary to said first direction and releases said distal pressure exerted on said cap.

Such embodiments allow locking the plunger rod in its connected configuration with the plunger stopper in a simple manner. The end user can therefore feel confident that the connection between the plunger rod and the plunger stopper is efficient and secure and that the injection step may be performed safely. Such embodiments further allow unlocking the plunger rod from the plunger stopper in a very simple manner.

In embodiments, the distal end of said rod is provided with an extension part configured to exert a radial force on said movable part to move said movable part from its second radial position to its first radial position when said rod moves back from its second axial position to its first axial position. Such an extension part may be located distally from said movable part and may be provided with inclined surfaces capable of cooperating with complementary inclined surfaces located on said movable part, in order to move the movable part in the centripetal direction when the extension part is moved in the proximal direction as the rod moves back from its second axial position to its first axial position.

The distal portion of the sleeve may be provided with more than one movable part; for example, the distal portion of the sleeve may be provided with two, three or four movable parts, that may be regularly distributed on a circumference of the sleeve. In embodiments, the distal portion of the sleeve is provided with two diametrically opposed movable parts. Such embodiments allow obtaining a secured and stable connection between the plunger rod and the plunger stopper, as two movable parts are anchored in the inner wall of the proximal cavity of the plunger stopper.

In embodiments, the plunger rod may further comprise a sheath dimensioned to surround said sleeve excepted said movable part, said sheath having an outer diameter substantially equal to the outer diameter of the distal portion of said sleeve when said movable part is in its first radial position. The sheath is preferably fixed in translation with respect to the sleeve. In such embodiments, the outer surface of the distal portion of the sleeve is flush with the outer surface of the sheath when the movable part(s) is/are in their first radial position: as such, the movable part(s) do not project outwardly radially from the sheath and the sleeve is therefore protected. Such embodiments are particularly convenient and user friendly, as the end user needs not taking care not to touch and/or damage the sleeve when handling the plunger rod.

In embodiments where the plunger rod comprises a sheath, the proximal end of said rod may be provided with a cap, and a proximal portion of the sheath may be provided with a radial flange, said biasing means may comprise a coil spring bearing on one hand on a proximal surface of said radial flange and on the other hand on a distal surface of said cap, said coil spring being capable of transitioning from a rest state where said rod is in its first axial position, to a stressed state where said rod is in its second axial position.

In such embodiments, the rod may be configured to rotate with respect to said sheath, said releasable locking means may comprise at least one hook extending distally from said cap and at least one partial circumferential groove located on said radial flange, said hook and said partial circumferential groove being configured to:
engage one another to lock said rod in its second axial position when one exerts a distal pressure on said cap so as to put said coil spring in its stressed state, and further rotates said rod with respect to said sheath in a first direction, and
disengage from one another to free said rod from its second axial position and move it automatically to its first axial position, when one rotates said rod with respect to said sheath contrary to said first direction and releases said distal pressure exerted on said cap.

In embodiments, said distal portion of said sleeve comprises at least one outwardly radially flexible leg, a distal end of said outwardly radially flexible leg forming said movable part. Alternatively, the distal portion of the sleeve may comprise two diametrically opposed outwardly radially flexible legs, a distal end of each outwardly radially flexible leg forming a movable part. Alternatively, the distal portion of the sleeve may comprise three outwardly radially flexible legs regularly distributed circumferentially, a distal end of each outwardly radially flexible leg forming a movable part. In such embodiments, the movable part may for example be in its first radial position when the flexible leg is in its rest state and the movable part may be in its second radial position when the flexible leg has been moved in the outwardly radially direction under the pressure applied by the distal end of the rod. When the distal end of the rod moves back to its first axial position, the flexible leg goes naturally back to its rest state, thereby causing the movable part to move automatically from its second radial position to its first radial position. Such embodiments are therefore very easy to manufacture and may be easily industrially implemented.

In embodiments, the movable part comprises an outer radial projection. Such outer radial projection is preferably configured to anchor in the inner wall of the proximal cavity under the centrifugal pressure exerted on the movable part by the distal end of the rod.

Another aspect of the invention is a method for connecting a plunger rod according as described above to the proximal cavity of a plunger stopper of an injection device, said method comprising the following steps:
providing the plunger rod with the rod in its first axial position,
inserting the distal portion of the sleeve inside the proximal cavity of the plunger stopper,
applying a distal pressure onto the proximal end of the rod to guide the rod in its second axial position,
activating the locking means to temporary lock the rod in its second axial position.

Another aspect of the invention is a method for disconnecting a plunger rod as described above which has been connected to the proximal cavity of a plunger stopper according to the connecting method above, comprising the following steps:
deactivating the locking means,
releasing all distal pressure applied on the proximal end of said rod to put said rod in its first axial position,
removing the distal portion of the sleeve from the proximal cavity of the plunger stopper.

### BRIEF DESCRIPTION OF THE DRAWINGS

The terms Fig., Figs., Figure, and Figures are used interchangeably in the specification to refer to the corresponding figures in the drawings.

The invention and the advantages arising therefrom will clearly emerge from the detailed description that is given below with reference to the appended drawings as follows:

FIG. 1 is a perspective view of a first embodiment of the plunger rod of the invention inserted into the plunger stopper of an injection device, before connection,

FIG. 2 is a cross section view of the plunger stopper and plunger rod of FIG. 1,

FIG. 3 is a detailed view of the plunger stopper and of the distal portion of the sleeve of the plunger rod of FIG. 2,

FIG. 4 is a detailed view of the proximal region of the plunger rod of FIG. 2,

FIG. 5 is a detailed perspective view of the distal portion of the sleeve of the plunger rod of FIG. 2,

FIG. 6 is a perspective view of the plunger stopper and plunger rod of FIG. 1, once the plunger rod is connected to the plunger stopper,

FIG. 7 is a cross section view of the plunger stopper and plunger rod of FIG. 6,

FIG. 8 is a detailed view of the plunger stopper and of the distal portion of the sleeve of the plunger rod of FIG. 6,

FIG. 9 is a detailed view of the proximal region of the plunger rod of FIG. 6,

FIG. 10 is a detailed view showing the locking means locking of the plunger rod of FIG. 6,

FIG. 11 is a cross section view of a second embodiment of the plunger rod of the invention inserted into a plunger stopper, before connection,

FIG. 12 is a detailed view of the plunger stopper and of the distal portion of the sleeve of the plunger rod of FIG. 11,

FIG. 13 is a perspective view of the plunger stopper and of the distal portion of the sleeve of the plunger rod of FIG. 12,

FIG. 14 is a cross section view of the plunger rod of FIG. 11 once connected into the plunger stopper,

FIG. 15 is a detailed view of view of the plunger stopper and of the distal portion of the sleeve of the plunger rod of FIG. 14,

FIG. 16 is a perspective view of the plunger stopper and of the distal portion of the sleeve of the plunger rod of FIG. 15,

FIG. 17 is a cross section view of a third embodiment of the plunger rod of the invention inserted into a plunger stopper, before connection,

FIG. 18 is a detailed view of the proximal region of the plunger rod of FIG. 17,

FIG. 19 is a perspective view of the distal portion of the sleeve and of the distal region of the sheath of the plunger rod of FIG. 17,

FIG. 20 is a detailed view of the distal portion of the sleeve and of the distal region of the sheath of the plunger rod of FIG. 17,

FIG. 21 is a cross section view of the plunger rod of FIG. 17 once connected into the plunger stopper,

FIG. 22 is a detailed view of the proximal region of the plunger rod of FIG. 17,

FIG. 23 is a detailed view showing the locking means of the plunger rod of FIG. 17,

FIG. 24 is a detailed view of the distal portion of the sleeve and of the distal region of the sheath of the plunger rod of FIG. 21,

FIG. 25 is a perspective view of the distal portion of the sleeve and of the distal region of the sheath of the plunger rod of FIG. 21.

### DESCRIPTION OF THE INVENTION

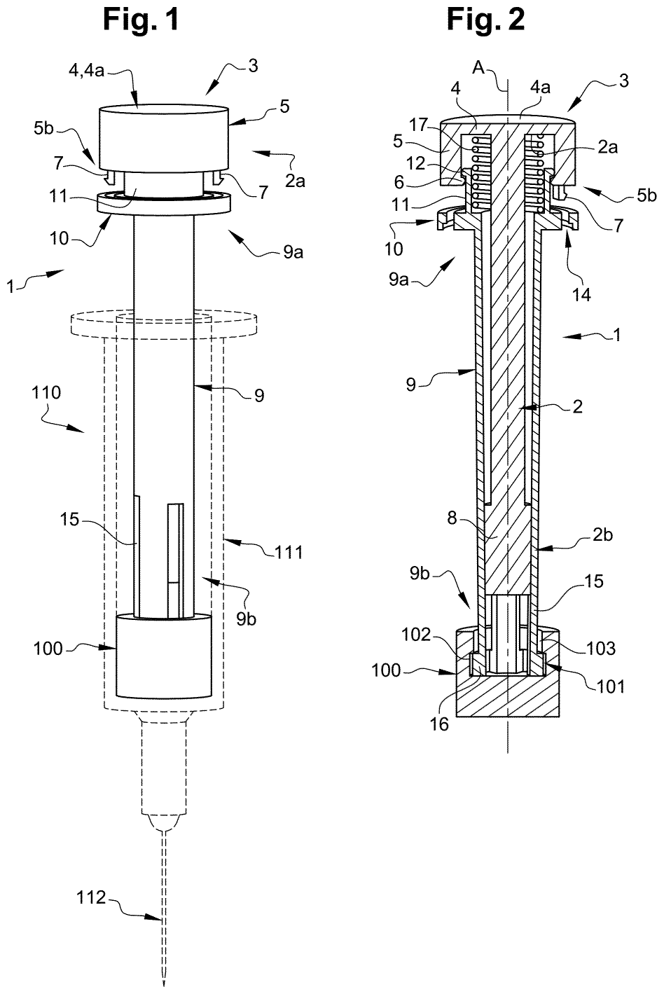

With reference to FIG. 1 is shown a plunger rod 1 of the invention intended to be connected to a plunger stopper 100. The plunger stopper 100 is intended to be inserted into the container 111 of an injection device 110 shown in dotted line in FIG. 1. The plunger stopper 100 is usually made of an elastomeric material, like rubber, and is used for sealing the proximal end of the container 111.

When the container 111 of the injection device contains a product to be injected, the plunger stopper 100 may be pushed distally in order to expel the product from the injection device 110 through the needle 112. When the container 111 is empty, as shown in FIG. 1, the plunger stopper 100 may be moved in the proximal direction to cause the needle 112 to suck a product stored in a separate vial into the container 111.

The plunger rod 1 is intended to be connected to the plunger stopper 100 in a secured manner in order to allow an end user to confidently move the plunger stopper 100 in the distal direction and/or in the proximal direction, depending on the intended use.

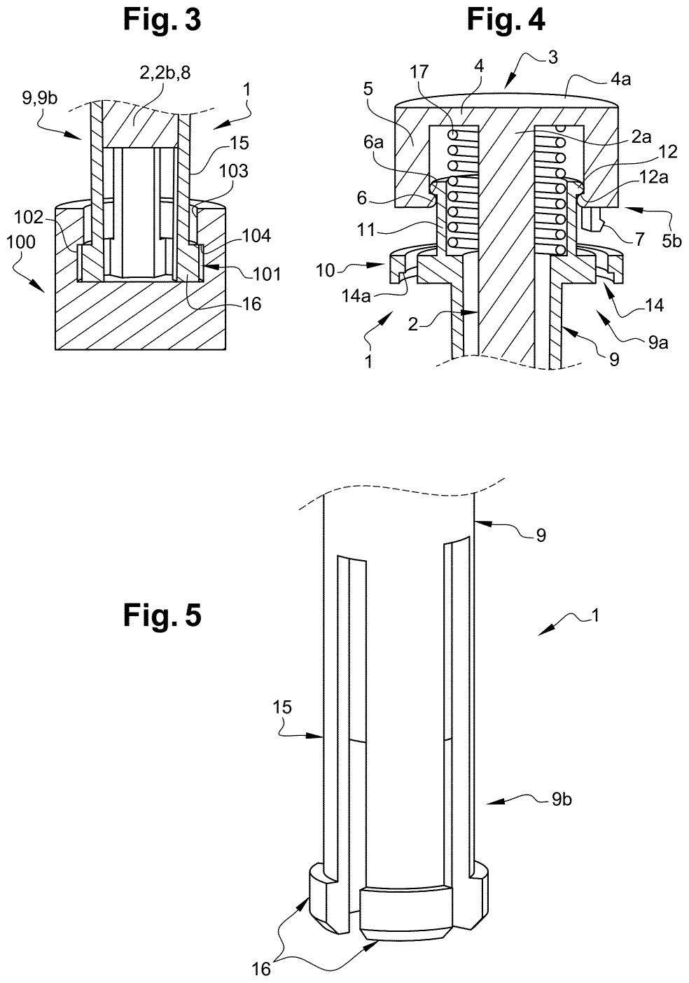

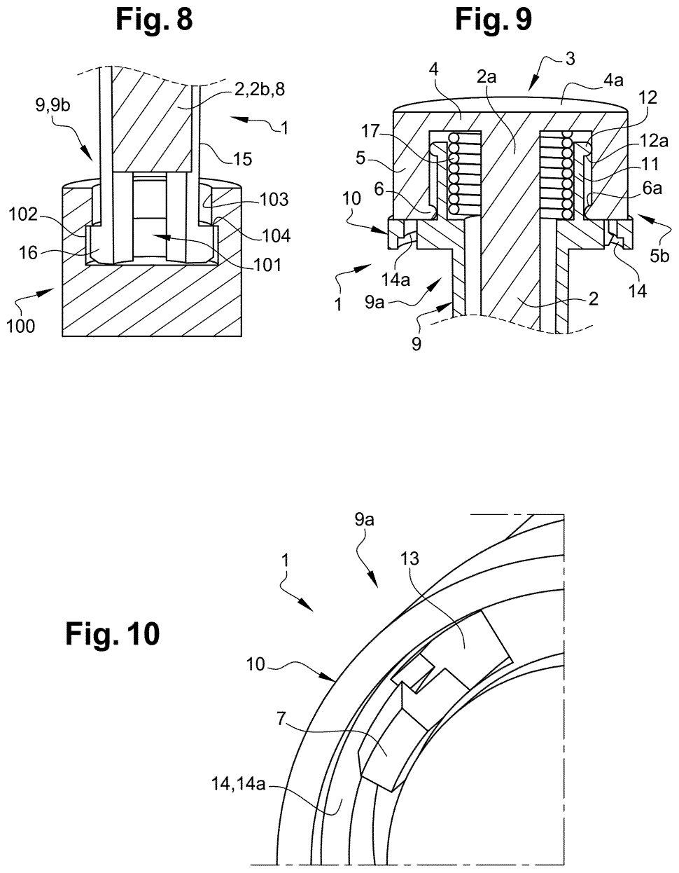

With reference to FIGS. 3 and 8, the plunger stopper 100 is provided with a proximal open cavity 101. Although not shown in the figures, the inner wall 102 of this proximal cavity may be provided with an inner thread, as is usually the case with commercially available plunger stoppers. In the example shown, the inner wall 102 of the proximal cavity 101 is provided with a proximal inner rib 103 forming an abutment surface 104.

With reference to FIGS. 1-10, the plunger rod 1 has a longitudinal axis A and comprises a rod 2 aligned on said axis. The rod 2 has a proximal end 2a and a distal end 2b. In the example shown, the proximal end 2a of the rod is provided with a cap 3 formed of a transversal wall 4 from which a tubular wall 5 extends in the distal direction. As will appear later in the description, the proximal face of the transversal wall 4 may form a pushing surface 4a for the end user to move the plunger rod 2 in the distal direction.

The tubular wall 5 is provided at its distal end 5b with an inner rim 6 forming a proximal abutment surface 6a. The distal end 5b of the tubular wall 5 is further provided with two (see FIG. 1) diametrically opposed distal hooks 7, the function of which will be explained later on.

In the example shown, the distal end 2b of the rod 2 is provided with an end part 8 showing an outer diameter of larger dimension than the outer diameter of the rest of the rod 2. In embodiments not shown, the distal end 2b of the rod 2 could show the same outer dimensions as the rest of the rod, as long as, as will appear below, the outer dimensions of the distal end 2b of the rod 2 allow moving the outer projections 16 of the flexible legs 15 of the sleeve 9 (see FIG. 7) in the centrifugal direction.

The plunger rod 2 further comprises a sleeve 9 having a proximal portion 9a and a distal portion 9b. The sleeve 9 is shaped and dimensioned to receive a distal region of the rod 2, and at least the distal end 2b of said rod 2. The proximal portion 9a of the sleeve 9 is provided with a radial flange 10 from which a tubular wall 11 extends in a proximal direction. The proximal end of the tubular wall 11 is provided with an outer rim 12 forming a distal abutment surface 12a.

The radial flange 10 is provided with two (only one being visible on FIG. 10) diametrically opposed through holes 13 dimensioned to allow the passage there through of the distal hooks 7, and with two adjacent partial circumferential grooves 14 forming distal abutment surfaces 14a.

In the example shown, the distal portion 9b of the sleeve 9 is provided with three flexible legs 15 capable of deflecting in the outward radial direction under the effect of a centrifugal force exerted thereon. The flexible legs 15 are regularly distributed along a circumference of the sleeve 9. In embodiments not shown, the number of flexible legs 15 could be less than three, such as one or two, or alternatively more than two, like for example four. The distal end of each flexible leg 15 is provided with an outer projection 16. Each outer projection 16 therefore forms a movable part capable of moving from a first radial position to a second radial position when a centrifugal force is applied on the flexible legs 15.

The plunger rod 2 is further provided with a coil spring 17 lodged between the radial flange 10 and the transversal wall 4 of the cap 3: the proximal end of the coil spring 17 bears on the distal face of the transversal wall 4 and the distal end of the coil spring 17 bears on the proximal face of the radial flange 10.

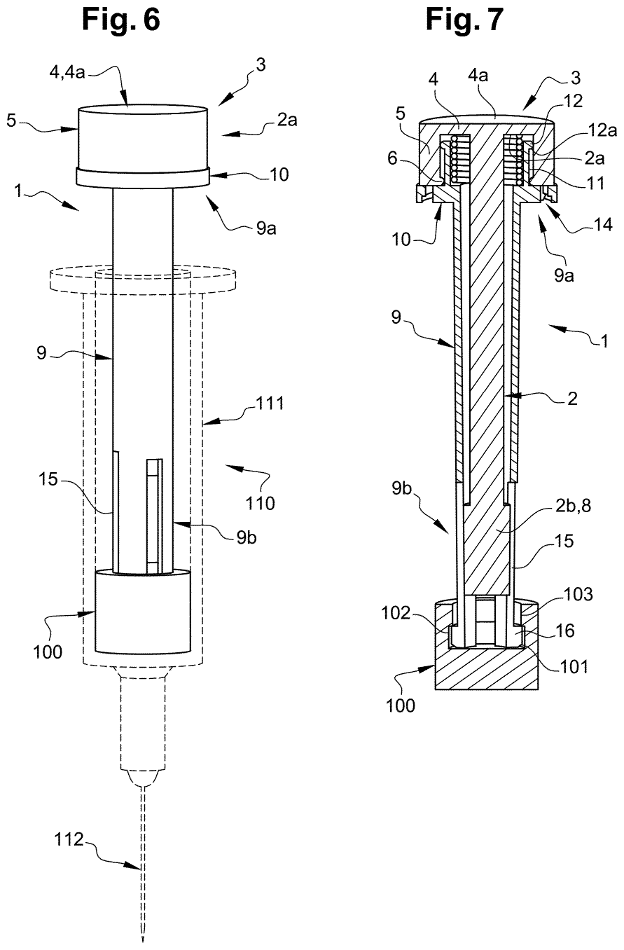

As will appear from the description below, the rod 2 is received in translation within the sleeve 9 and is capable of translating from a first axial position, as shown in FIGS. 1-5 to a second axial position, distally spaced from said first axial position, as shown in FIGS. 6-10, under the effect of a distal pressure exerted on the pushing surface 4a by an end user.

The connection of the plunger rod 1 to the plunger stopper 100 will now be described in reference to FIGS. 1-10.

With reference to FIGS. 1-5, the plunger rod 1 is not connected to the plunger stopper 100 and the rod 2 is in its first axial position. In such a position, the coil spring 17 is in a rest state, the distal end of the tubular wall 5 of the cap 3 is not in contact with the proximal face of the radial flange 10, and the coil spring 17 couples the rod 2 to the sleeve 9 by means of the distal abutment surface 12a of the outer rim 12 of the tubular wall 11 of the radial flange 10 resting on the proximal abutment surface 6a of the inner rim 6 of the tubular wall 5 of the cap 3 (see FIG. 4). In this position, the distal end 2b of the rod 2 is not in contact with the distal ends of the flexible legs 15 and these flexible legs 15 are in a rest state. As a consequence, the outer projections 16 are in their first radial position. As a result, the outer dimensions of the distal portion 9b of the sleeve 9 are smaller than the inner dimensions of the inner wall 102 of the proximal open cavity 101 of the plunger stopper 100. The distal portion 9b of the sleeve 9 is therefore easily inserted in the proximal open cavity 101 of the plunger stopper 100, as can be seen in FIGS. 2 and 3.

Once the distal portion 9b of the sleeve 9 is inserted in the proximal open cavity 101 of the plunger stopper 100, the end user who wishes to connect the plunger rod 1 to the plunger stopper 100 simply applies a distal pressure onto the pushing surface 4a of the transversal wall 4 of the cap 3 of the plunger rod 2. Under this distal pressure, the distal end of the tubular wall 5 of the cap 3 comes in contact with the proximal face of the radial flange 10 and the coil spring 17 transitions to its stressed state. The distal movement of the rod 2 has caused the rod 2 to move from its first axial position to its second axial position, as shown in FIGS. 6-10. The end part 8 of the distal end 2b of the rod 2 has come in contact with the distal ends of the flexible legs 15 and has applied a centrifugal force onto the distal ends of these flexible legs 15, causing these flexible legs 15 to deflect outwardly radially. This has caused the outer projections 16 to move from their first radial position to their second radial position, as shown in FIG. 8. In this second radial position, each outer projection 16 harpoons the inner wall 102 of the proximal open cavity 101 of the plunger stopper 100, thereby realizing a safe connection of the plunger rod 2 to the plunger stopper 100. Thanks to the centrifugal pressure exerted by the end part 8 onto the outer projections 16, the outer projections 16 anchor into the inner wall 102 of the proximal open cavity 101, regardless of the design of this inner wall 102, and in particular, independently from any inner thread that may be present on said inner wall 102. The efficiency of the connection may be reinforced by the presence of the abutment surface 104 of the inner rim 103 of the plunger stopper 100, said abutment surface 104 preventing the outer projections 16 to move in the proximal direction.

When the distal end of the tubular wall 5 of the cap 3 has come in contact with the proximal face of the radial flange 10, each distal hook 7 has passed through the corresponding through hole 13. As a consequence, in order to lock the rod 2 in its second axial position, the end user needs only to rotate the cap 3 with respect to the sleeve 9 in a first direction in order to bring the distal hooks 7 in contact with the abutment surfaces 14a of the partial circumferential grooves 14, as shown in FIG. 10. The coil spring 17 in its stressed state and the distal hooks 7 engaged in the abutment surfaces 14a of the partial circumferential grooves 14 form releasable locking means of the rod 2 in its second axial position. The end user can feel confident that the connection between the plunger rod 1 and the plunger stopper 100 is efficient and secured and he can manipulate the plunger rod 1 safely in order to move the plunger stopper 100 within the container 111 of the injection device 110.

When the end user wishes to disconnect the plunger rod 1 from the plunger stopper 100, he simply needs to rotate the cap 3 with respect to the sleeve 9 contrary to the first direction previously used in order to disengage the distal hooks 7 from the partial circumferential grooves 14. The end user then releases the distal pressure he exerts on the pushing surface 4a of the cap 3 and the rod 2 comes automatically back to its first axial position, under the effect of the coil spring 17 naturally transitioning to its rest state, as shown in FIG. 2. During this operation, the end part 8 of the distal end 2b of the rod 2 has left the distal ends of the flexible legs 15 which have come back to their rest state. The outer projections 16 have therefore moved from their second radial position to their first radial position, as shown in FIG. 3, and the plunger rod 1 may easily be disconnected from the plunger stopper 100 by simply removing the distal portion 9b of the sleeve 9 from the proximal open cavity 101 of the plunger stopper 100.

FIGS. 11-16 relate to a second embodiment of the plunger rod 1 of the invention, in which the flexible legs and the outer projections are replaced by non flexible movable parts; The elements which are identical to that of the plunger rod 1 of FIGS. 1-10 are designated by the same references.

With reference to these figures, the distal end of the rod 2 is provided with an extension part 18 showing a hollow conical surface 18a extending in the proximal direction. The distal region of the rod 2 further comprises an outer conical part 19, tapering in the distal direction, said outer conical part 19 being located proximal to the extension part 18. The sleeve 9 receives in its distal portion 9b two diametrically opposed mobile parts 20, each mobile part 20 comprising a proximal inclined surface 20a and a distal inclined surface 20b. The wall of the sleeve 9 is provided with two diametrically opposed windows 21, each window 21 facing a mobile part 20. The mobile parts 20 and the windows 21 of the sleeve 9 are axially located between the extension part 18 and the outer conical part 19 of the rod 2.

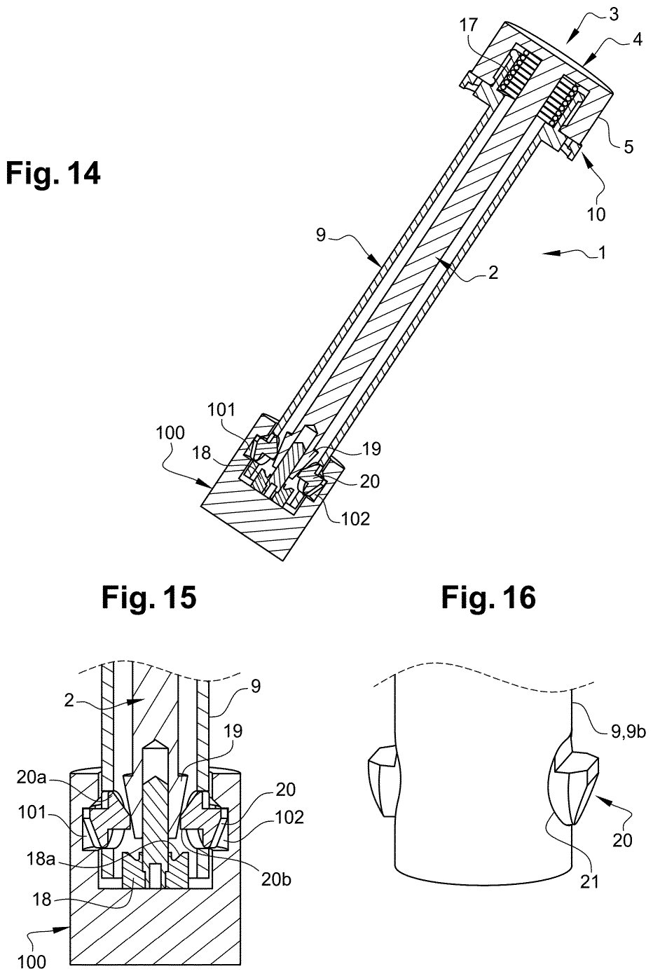

The rod 2 is shown in its first axial position in FIGS. 11-13, and in its second axial position in FIGS. 14-16.

With reference to FIGS. 11-13, the outer conical part 19 of the rod 2 is not in contact with the mobile parts 20, which are entirely located within the walls of the sleeve 9. The outer dimensions of the distal portion 9b of the sleeve 9 are less than the inner dimensions of the proximal open cavity 101 of the plunger stopper 100.

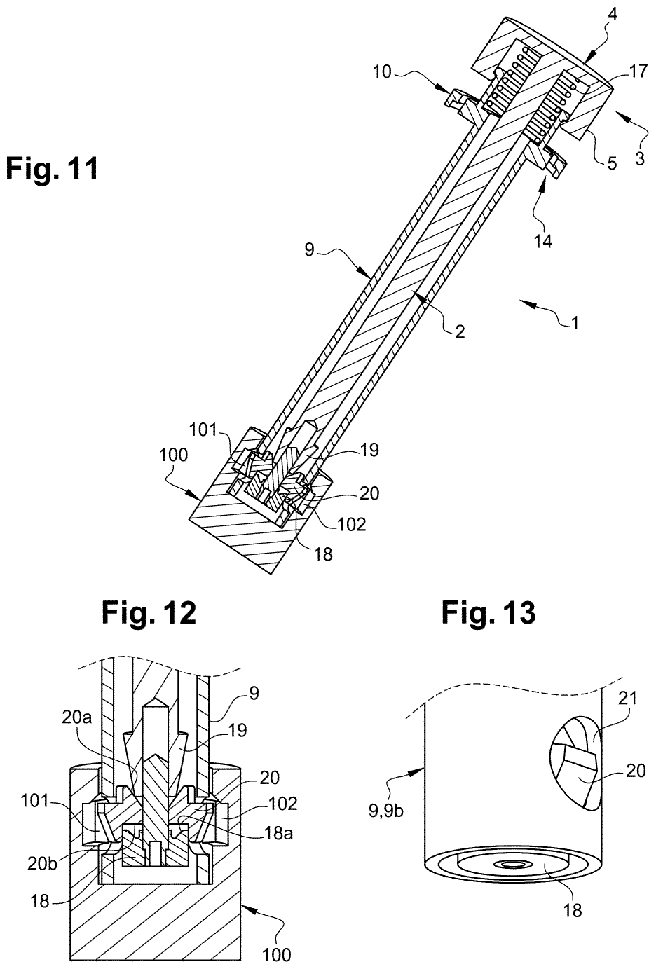

When an end user wishes to connect the plunger rod 1 to the plunger stopper 100, he applies a pressure onto the transversal wall 4 of the cap 3 of the plunger rod 1 as described above. When the rod 2 moves to its second axial position, as shown in FIGS. 14-16, the outer conical part 19 comes in contact with the proximal inclined surfaces 20a of the mobile parts 20 and applies a centrifugal force on these surfaces, thereby causing the mobile parts 20 to move in the centrifugal direction. The mobile parts 20 therefore move from their first radial position, as shown in FIGS. 12 and 13, to their second radial position, as shown in FIGS. 15 and 16. The mobile parts 20 therefore projects out from the windows 21 of the wall of the sleeve 9 and they harpoon the inner wall 102 of the proximal open cavity 101 of the plunger stopper, thereby realizing the connection of the plunger rod 1 to the plunger stopper 100.

In this position, the end user may lock the rod 2 in its second axial position in the same manner as described above with reference to FIGS. 1-10.

When the end user wishes to disconnect the plunger rod 1 from the plunger stopper 100, he releases the coil spring 17 as described above in FIGS. 1-10.

When the rod 2 moves proximally from its second axial position to its first axial position, the extension part 18 moves equally proximally and its hollow conical surface 18a comes in contact with the distal inclined surfaces 20b of the mobile parts 20 (see FIG. 15). The hollow conical surface 18a slides onto the distal inclined surfaces 20b of the mobile parts 20, thereby applying a centripetal force on theses surfaces and causing the mobile parts 20 to move backwards in the direction of their first radial position. As a consequence, the mobile parts 20 withdraw within the interior of the sleeve 9, as shown in FIG. 13. The end user may therefore easily disconnect the plunger rod 1 from the plunger stopper 100 by removing the distal portion 9b of the sleeve 9 from the proximal open cavity 101 of the plunger stopper 100.

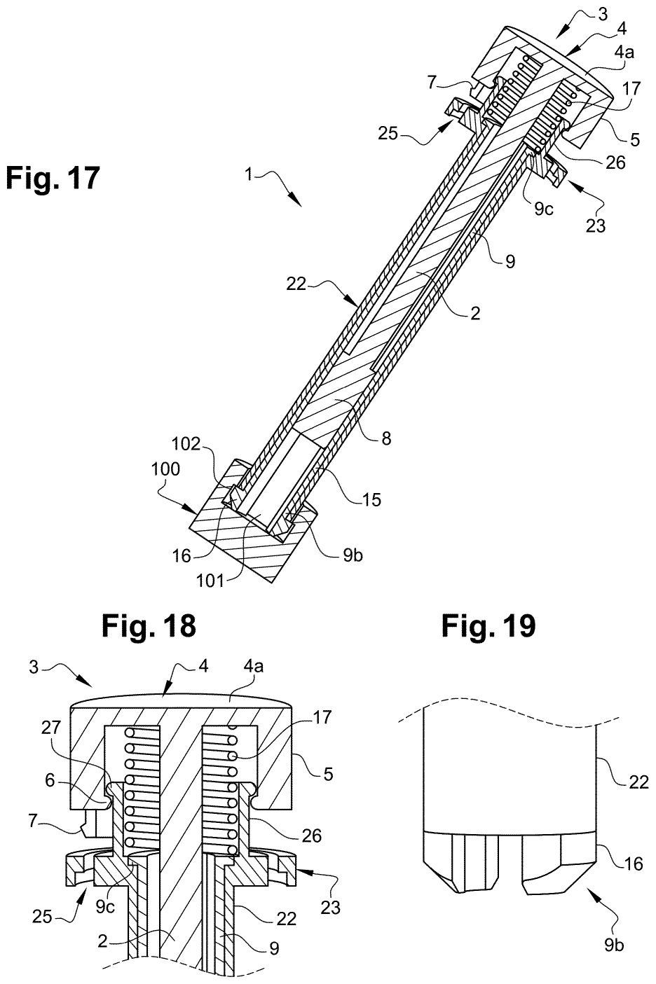

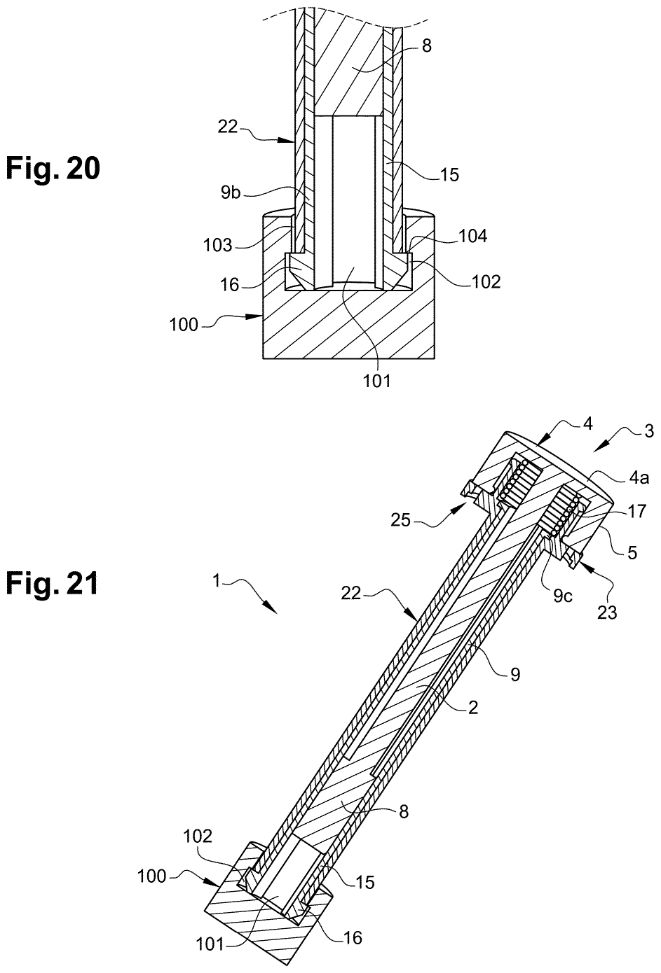

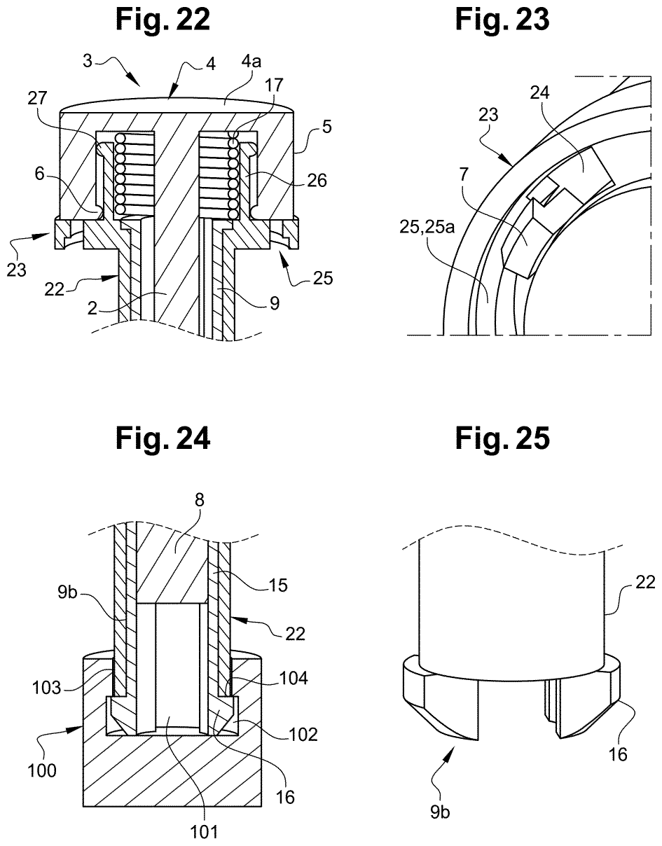

FIGS. 17-25 relate to a third embodiment of the plunger rod 1 of the invention, similar to that of FIGS. 1-10, but additionally comprising an outer sheath surrounding the sleeve. The elements which are identical to that of the plunger rod 1 of FIGS. 1-10 are designated by the same references.

With reference to these figures, the plunger rod 1 comprises a rod 2 identical to that of FIGS. 1-10, comprising a cap 3 and an end part 8. The plunger rod 1 further comprises a sleeve 9, the distal portion 9b of which is provided with two diametrically opposed flexible legs 15. The distal end of each flexible leg 15 is provided with an outer projection 16. Contrary to the embodiment of FIGS. 1-10, the proximal end of the sleeve 9 is free of any radial flange and comprises an outer rim 9c.

The plunger rod 1 of FIGS. 17-25 further comprises an outer sheath 22 surrounding the sleeve 9, except for the outer projections 16. The proximal end of the outer sheath 22 is provided with a radial flange 23, to which is fixed the outer rim 9c of the sleeve 9. The sleeve 9 and the outer sheath 22 are fixed to each other. With reference to FIG. 19, the outer sheath 22 has an outer diameter substantially equal to the outer diameter of the distal portion 9b of the sleeve 9 when the outer projections 16 are in their first radial position.

The radial flange 23 is similar to the radial flange 10 of embodiments of FIGS. 1-10, except that it is provided at the proximal end of the outer sheath 22 rather than at the proximal end of the sleeve 9. As a consequence, the radial flange 23 is provided with two (only one being visible on FIG. 23) diametrically opposed through holes 24 dimensioned to allow the passage there through of the distal hooks 7, and with two adjacent partial circumferential grooves 25 forming distal abutment surfaces 25a. The radial flange 23 is further provided with a tubular wall 26 extending in the proximal direction. The proximal end of the tubular wall 26 is provided with an outer rim 27 intended to cooperate with the inner rim 6 of the cap 3 in order to maintain the coil spring 17 in its rest state (see FIG. 18).

The method for connecting the plunger rod 1 of FIGS. 17-25 is the same as that described above for the embodiment of FIGS. 1-10. The plunger rod 1 of FIGS. 17-25 has the advantage that the sleeve 9 is protected by the outer sheath 22 during the connecting and disconnecting operations. In particular, as visible on FIG. 19, the outer surface of the outer projections 16 is flush with the outer surface of the outer sheath 22. This is very convenient for the end user who needs not taking care not to touch the sleeve and the outer projections when he manipulates the plunger rod 1.

With reference to FIGS. 17-20, the plunger rod 1 is not connected to the plunger stopper 100 and the rod 2 is in its first axial position. In such a position, the coil spring 17 is in a rest state, the distal end of the tubular wall 5 of the cap 3 is not in contact with the proximal face of the radial flange 23, and the coil spring 17 couples the rod 2 to the outer sheath 22 by means of the outer rim 27 of the tubular wall 26 of the radial flange 23 resting on the inner rim 6 of the tubular wall 5 of the cap 3 (see FIG. 18). In this position, the distal end 2b of the rod 2 is not in contact with the distal ends of the flexible legs 15 and these flexible legs 15 are in a rest state. As a consequence, the outer projections 16 are in their first radial position, as shown in FIG. 19. The outer dimensions of the distal portion 9b of the sleeve 9 and of the distal portion of the outer sheath 22 are smaller than the inner dimensions of the inner wall 102 of the proximal open cavity 101 of the plunger stopper 100. The distal portion 9b of the sleeve 9 is therefore easily inserted in the proximal open cavity 101 of the plunger stopper 100, as can be seen in FIG. 17.

As previously described, when the end user then applies a distal pressure on the cap 3, the coil spring 17 transitions to its stressed state, and the end part 8 of the distal end 2b of the rod 2 comes in contact with the distal ends of the flexible legs 15 and applies a centrifugal force onto the distal ends of these flexible legs 15, causing these flexible legs 15 to deflect outwardly radially. The outer projections 16 to move from their first radial position to their second radial position, where they project out of the outer sheath 22, as shown in FIG. 25. Thanks to the centrifugal pressure exerted by the end part 8 onto the outer projections 16, the outer projections 16 anchor into the inner wall 102 of the proximal open cavity 101, as shown in FIG. 24, regardless of the design of this inner wall 102, and in particular, independently from any inner thread that may be present on said inner wall 102. The efficiency of the connection may be reinforced by the presence of the abutment surface 104 of the inner rim 103 of the plunger stopper 100, said abutment surface 104 preventing the outer projections 16 to move in the proximal direction.

The locking of the rod 2 in this second axial position (shown in FIGS. 21-25) may be performed similarly as previously described for the embodiment of FIGS. 1-10. To do so, the end user needs only to rotate the cap 3 with respect to the outer sheath 22 in a first direction in order to bring the distal hooks 7 in contact with the abutment surfaces 25a of the partial circumferential grooves 25, as shown in FIG. 25. The end user can feel confident that the connection between the plunger rod 1 and the plunger stopper 100 is efficient and secured and that he can manipulate the plunger rod 1 safely in order to move the plunger stopper 100 within the container 111 of the injection device 110.

In order to disconnect the plunger rod 1 from the plunger stopper 100, the end user simply rotates the cap 3 with respect to the outer sheath 22 contrary to the first direction previously used in order to disengage the distal hooks 7 from the partial circumferential grooves 25. The end user then releases the distal pressure he exerts on the pushing surface 4a of the cap 3 and the rod 2 comes automatically back to its first axial position, under the effect of the coil spring 17 naturally transitioning to its rest state, as shown in FIG. 17. During this step, the flexible legs 15 have come back to their rest state, and the outer projections 16 have come back to their first radial position, as shown in FIG. 19. The end user can therefore easily disconnect the plunger rod 1 from the plunger stopper 100 by removing the distal portion of the outer sheath 22 from the proximal cavity 101 of the plunger stopper 100.

The plunger rod of the invention therefore allows simplifying the connecting step of a plunger rod to a plunger stopper as a screwing step is no longer necessary. In addition, the plunger rod of the invention may be connected safely to any plunger stopper, whatever the material of the plunger stopper and the design of the inner wall of the cavity of the plunger stopper. A single plunger rod may be used for a plurality of plunger stoppers and/or injections devices, thereby helping reducing the plastic waste generated in the sector of medical devices.

1. A plunger rod intended to be connected to a proximal cavity of a plunger stopper of an injection device, said plunger rod comprising:
a rod having a proximal end and a distal end,
a sleeve configured to receive at least the distal end of the rod, a distal portion of said sleeve being provided with at least one movable part capable of moving from a first radial position to a second radial position under the effect of a force applied on said movable part,
the rod being capable of sliding in translation within said sleeve from a first axial position, in which the distal end of said rod is not in contact with said movable part, said movable part is in its first radial position and the distal portion of said sleeve may freely enter in, and exit from, said proximal cavity of said plunger stopper, to a second axial position, in which the distal end of said rod has applied a force on said movable part to move said part in its second radial position, and said movable part is capable of harpooning an inner wall of said proximal cavity of said plunger stopper to connect said plunger rod to said plunger stopper, said rod being configured to translate from its first axial position to its second axial position under the effect of a distal pressure exerted on its proximal end, and
a releasable lock configured for temporary locking said rod in its second axial position.

2. The plunger rod according to claim 1, further comprising a biasing member configured for automatically moving said rod from its second axial position to its first axial position when the releasable lock is released.

3. The plunger rod according to claim 1, wherein the proximal end of said rod is being provided with a button cap, and a proximal portion of the sleeve is being provided with a radial flange, and wherein said biasing member comprise a coil spring abutting on both a proximal surface of said radial flange and on a distal surface of said cap, said coil spring being capable of transitioning from a rest position where said rod is in its first axial position, to a stressed state where said rod is in its second axial position.

4. The plunger rod according to claim 3, wherein said rod is being configured to rotate with respect to said sleeve, said releasable lock comprising at least one hook extending distally from said cap and at least one partial circumferential groove located on said radial flange, said hook and said circumferential groove being configured to:
engage one another to lock said rod in its second axial position when one exerts a distal pressure on said cap so as to put said coil spring in its stressed state, and further rotates said rod with respect to said sleeve in a first direction, and
disengage from one another to free said rod from its second axial position and move it automatically to its first axial position, when one rotates said rod with respect to said sleeve contrary to said first direction and releases said distal pressure exerted on said cap.

5. The plunger rod according to claim 1, wherein the distal portion of said sleeve is provided with two diametrically opposed movable parts.

6. The plunger rod according to claim 1, wherein the distal end of said rod is provided with an extension part configured to exert a radial force on said movable part to move said movable part from its second radial position to its first radial position when said rod moves back from its second axial position to its first axial position.

7. The plunger rod according to claim 1, further comprising a sheath dimensioned to surround said sleeve excepted said movable part, said sheath having an outer diameter substantially equal to the outer diameter of the distal portion of said sleeve when said movable part is in its first radial position.

8. The plunger rod according to claim 1, wherein the proximal end of said rod is provided with a cap, and a proximal portion of the sheath is provided with a radial flange, said biasing member comprises a coil spring bearing on one hand on a proximal surface of said radial flange and on the other hand on a distal surface of said cap, said coil spring being capable of transitioning from a rest state where said rod is in its first axial position, to a stressed state where said rod is in its second axial position.

9. The plunger rod according to the claim 8 wherein said rod is configured to rotate with respect to said sheath, said releasable lock comprises at least one hook extending distally from said cap and at least one partial circumferential groove located on said radial flange, said hook and said partial circumferential groove being configured to:
engage one another to lock said rod in its second axial position when one exerts a distal pressure on said cap so as to put said coil spring in its stressed state, and further rotates said rod with respect to said sheath in a first direction, and
disengage from one another to free said rod from its second axial position and move it automatically to its first axial position, when one rotates said rod with respect to said sheath contrary to said first direction and releases said distal pressure exerted on said cap.

10. The plunger rod according to claim 1, wherein said distal portion of said sleeve comprises at least one outwardly radially flexible leg, a distal end of said outwardly radially flexible leg forming said movable part.

11. The plunger rod according to claim 10, wherein said distal portion of said sleeve comprises two diametrically opposed outwardly radially flexible legs, a distal end of each outwardly radially flexible leg forming a movable part.

12. The plunger rod according to claim 10, wherein said distal portion of said sleeve comprises three outwardly radially flexible legs regularly distributed circumferentially, a distal end of each outwardly radially flexible leg forming a movable part.

13. The plunger rod according to claim 1, wherein the movable part comprises an outer radial projection.

14. A method for connecting a plunger rod according to claim 1 to the proximal cavity of a plunger stopper of an injection device, said method comprising the following steps:
providing the plunger rod with the rod in its first axial position,
inserting the distal portion of the sleeve-inside the proximal cavity of the plunger stopper,
applying a distal pressure onto the proximal end of the rod to guide the rod in its second axial position, and
activating the releasable lock to temporary lock the rod in its second axial position.

15. The method according to claim 14, further comprising disconnecting the plunger rod from the plunger stopper comprising the following steps:
deactivating the releasable lock,
releasing all distal pressure applied on the proximal end of said rod to put said rod in its first axial position, and
removing the distal portion of the sleeve from the proximal cavity of the plunger stopper.
